(19) United States
(12) Patent Application Publication
(10) Pub. No.: US 2026/0144720 A1
(43) Pub. Date: May 28, 2026

(54) COSMETIC COMPOSITIONS CONTAINING PIGMENT PARTICLES IN A HYDROPHOBIC PHASE

(71) Applicant: ELC Management LLC, Melville, NY (US)

(72) Inventors: John Richard Castro, Little Neck, NY (US); Liangchen Xu, West Babylon, NY (US)

(73) Assignee: ELC Management LLC, Melville, NY (US)

(21) Appl. No.: 18/960,508

(22) Filed: Nov. 26, 2024

(51) Int. Cl. A61K 8/06

(52) U.S. Cl. CPC A61K 8/064; A61Q 19/00; A61K 2800/28; A61K 2800/30; A61K 2800/43; A61K 2800/621

(57) ABSTRACT

Cosmetic compositions for application to the skin comprise: a dispersion medium comprising an aqueous phase and a hydrophobic phase; a carboxylic acid-based agent comprising at least one carboxylic acid, any ester thereof, any salt thereof, or any combination thereof dispersed in the aqueous phase of the dispersion medium; and a plurality of coated pigment particles dispersed as solids in the hydrophobic phase of the dispersion medium. For example, the coated pigment particles may comprise a silica coating.

### FIELD

The present disclosure generally relates to cosmetic compositions, and more specifically, to cosmetic compositions containing dispersed pigment particles.

### BACKGROUND

As people age, the skin undergoes many types of changes. The top layer of skin, known as the stratum corneum, is composed primarily of cells that are mainly composed of dehydrated keratin protein. In a normal process called differentiation, epidermal cells are gradually pushed to the surface by underlying cells, where they undergo exfoliation. In abnormal processes not leading to exfoliation, the epidermal cells accumulate on the surface and form hyperkeratotic tissue, which may cause discomfort. Hyperkeratotic tissue in mammals includes tylomas (calluses and fissures), helomas (corns), keratoses (papules), and dry skin scales and flakes, including dandruff.

Exfoliation refers to the removal of flaky corneocytes that are ready to detach from the stratum corneum, which may promote smoother, less flaky skin. Exfoliation improves skin cleansing by promoting mechanical removal of dirt, oil, and microorganisms from the skin. Other potential benefits of exfoliation may include increased blood flow to the skin due to the mechanical stimulation. Common keratolytic agents for promoting release of hyperkeratinized tissue through exfoliation include, for example, salicylic acid, retinoic acids, alpha-hydroxyacids (e.g., lactic acid and glycolic acid), beta-hydroxyacids, and polyhydroxyacids. Polyhydroxyacids are often particularly desirable keratolytic agents due to their minimal skin irritation.

Keratolytic agents may be included in various types of cosmetic compositions, such as concealer, foundation, and the like. Foundation refers to a cosmetic composition that supplies a tinted film to the skin to promote skin smoothness and color correction. Pigments may be included in a foundation in a suitable amount to provide the tinting and color correction. However, the low pH values associated with carboxylic acid-based keratolytic agents may lead to issues of pigment stability issues such as separation, discoloration, and settling. Therefore, it may be difficult to provide coloration of foundations and other cosmetic compositions containing hydroxyacids as keratolytic agents.

### SUMMARY

In some embodiments, the present disclosure provides cosmetic compositions comprising: a dispersion medium comprising an aqueous phase and a hydrophobic phase; a carboxylic acid-based agent comprising at least one carboxylic acid, any ester thereof, any salt thereof, or any combination thereof dispersed in the aqueous phase of the dispersion medium; and a plurality of coated pigment particles dispersed as solids in the hydrophobic phase of the dispersion medium.

In some or other embodiments, the present disclosure provides cosmetic compositions comprising: a dispersion medium comprising an aqueous phase and a hydrophobic base, the aqueous phase having a pH ranging from about 5 to about 5.5; wherein the dispersion medium comprises a water-in-oil emulsion comprising the aqueous phase as a dispersed phase and the hydrophobic phase as a continuous phase; about 1 wt % to about 10 wt % of gluconic acid, any ester thereof, any salt thereof, or any combination thereof dispersed in the aqueous phase of the dispersion medium, based upon total mass of the cosmetic composition; about 2 wt % to about 20 wt % of a plurality of coated pigment particles dispersed as solids in the hydrophobic phase of the dispersion medium, based upon total mass of the cosmetic composition; wherein the coated pigment particles comprise a silica coating; and about 1 wt % to about 10 wt % of niacinamide dispersed in the aqueous phase of the dispersion medium, based upon total mass of the cosmetic composition.

In still other embodiments, the present disclosure provides methods for preparing cosmetic compositions comprising: providing a first dispersion comprising a plurality of coated pigment particles dispersed as solids in a hydrophobic phase; wherein the coated pigment particles comprise a silica coating; blending the first dispersion with a second dispersion comprising a carboxylic acid-based agent, any ester thereof, any salt thereof, or any combination thereof dispersed in an aqueous phase; wherein the second dispersion has a pH ranging from about 4 to about 9; and obtaining a dispersion medium comprising the aqueous phase as a dispersed phase and the hydrophobic phase as a continuous phase; wherein, in the dispersion medium, the carboxylic acid-based agent, any ester thereof, any salt thereof, or any combination thereof remains in the aqueous phase, and the coated pigment particles remain dispersed as solids in the hydrophobic phase.

### BRIEF DESCRIPTION OF THE DRAWINGS

Not applicable.

### DETAILED DESCRIPTION

The present disclosure generally relates to cosmetic compositions, and more specifically, to cosmetic compositions containing dispersed pigment particles.

As discussed above, it may be difficult to formulate pigments in the presence of carboxylic acid-based keratolytic agents, such as in foundations or similar cosmetic compositions, due to the low pH values. In response to the foregoing issue, the present disclosure provides cosmetic compositions having increased pigment particle stability, as well as other related advantages.

In particular, cosmetic compositions of the present disclosure may comprise: a dispersion medium comprising an aqueous phase and a hydrophobic phase; a carboxylic acid-based agent comprising at least one carboxylic acid, any ester thereof, any salt thereof, or any combination thereof dispersed in the aqueous phase of the dispersion medium; and a plurality of coated pigment particles dispersed as solids in the hydrophobic phase of the dispersion medium.

In the present disclosure, the term “cosmetic composition” refers to any formulation that is applied to a bodily surface, such as a skin surface, for achieving a specified purpose. The specified purpose is not considered to be particularly limited. The specified purpose may be to achieve a desired appearance, to promote healthy skin, and/or to achieve a desired therapeutic or preventative effect. In non-limiting examples, the cosmetic compositions of the present disclosure may define a foundation, a skin conditioner, a skin treatment, or any combination thereof.

In the present disclosure, the term “dispersed” refers to the distribution of a substance throughout a medium. A substance may be “dispersed” in a medium as a plurality of insoluble particles distributed uniformly or near-uniformly throughout the medium, or the substance may be dissolved or partially dissolved in the medium. Solids and emulsified liquids may be dispersed by being suspended as particles or droplets in the medium. In the cosmetic compositions of the present disclosure, the coated pigment particles are dispersed as solids in the hydrophobic phase of the dispersion medium, and the carboxylic acid-based agent is at least partially dissolved in the aqueous phase of the dispersion medium, preferably dissolved as a solution in the aqueous phase.

The dispersion medium in the cosmetic compositions of the present disclosure may comprise the aqueous phase as a dispersed phase of the dispersion medium and the hydrophobic phase as a continuous phase of the dispersion medium. The dispersion medium may be in the form of an emulsion. The emulsion may comprise a water-in-oil emulsion having the aqueous phase as a dispersed phase of the dispersion medium and the hydrophobic phase as a continuous phase of the dispersion medium. As used herein, the term “phase” and grammatical variants thereof, refers to a state having a phase boundary between two media and any substances present therein. For example, in the present disclosure, the aqueous phase may comprise water, optional water-miscible solvents, and any additives localized therein, such as a dissolved or dispersed carboxylic acid-based agent.

The dispersion medium in the cosmetic compositions of the present disclosure may comprise the aqueous phase and the hydrophobic phase in any ratio effective to promote stable formulation of the coated pigment particles and the carboxylic acid-based agent. In non-limiting examples, the dispersion medium may have an aqueous phase:hydrophobic phase volume ratio ranging from about 7:3 to about 1:99, or about 1:1 to about 1:99, or about 1:4 to about 1:99, or about 1:3 to about 1:99, or about 7:3 to about 1:1, or about 7:3 to about 1:4, or about 7:3 to about 1:3, or about 1:1 to about 1:9, or about 1:4 to about 1:9, or about 1:3 to about 1:9, or about 1:1 to about 1:3, or about 1:1 to about 1:4.

In non-limiting examples, the dispersion medium may comprise the aqueous phase in an amount ranging from about al to about a2 wt %, based on total mass of the cosmetic composition, where a1 and a2 may be, independently, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60, and where a1<a2. For example, the cosmetic compositions may comprise the aqueous phase in an amount ranging from about 20 wt % to about 30 wt %, or about 30 wt % to about 40 wt %, or about 40 wt % to about 50 wt %, or about 50 wt % to about 60 wt %, or about 20 wt % to about 40 wt %, or about 30 wt % to about 50 wt %, or about 40 wt % to about 60 wt %, or about 20 wt % to about 50 wt %, or about 30 wt % to about 60 wt %, or about 20 wt % to about 60 wt %, based on total mass of the cosmetic compositions.

In some or other examples, the cosmetic compositions may comprise the hydrophobic phase in an amount ranging from about bl to about b2, based on total mass of the cosmetic composition, where b1 and b2 may be, independently, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80, and where b1<b2. For example, the cosmetic compositions may comprise the hydrophobic phase in an amount ranging from about 40 wt % to 50 wt %, 50 wt % to about 60 wt %, or about 60 wt % to about 70 wt %, or about 70 wt % to about 80 wt %, or about 40 wt % to about 60 wt %, or about 50 wt % to about 70 wt %, or about 60 wt % to about 80 wt %, or about 40 wt % to about 70 wt %, or about 50 wt % to about 80 wt %, or about 40 wt % to about 80 wt %.

Hydrophobic phases suitable for use in the cosmetic compositions of the present disclosure may comprise one or more hydrophobic substances. Suitable hydrophobic substances may be substantially non-irritating to the skin and may be in a liquid state at room temperature. The hydrophobic substances may be volatile or nonvolatile. As used herein, the term “nonvolatile” refers to a material that exhibits a vapor pressure of no more than about 0.2 mm Hg at 25° C. at one atmosphere and/or a material that has a boiling point at one atmosphere of at least about 300° C. As used herein, the term “volatile” refers to a material that exhibits a vapor pressure of at least about 0.2 mm of mercury at 20° C. Hydrophobic substances that are volatile may be used to provide a lighter feel when a heavy, greasy film is undesirable. In the cosmetic compositions disclosed herein, the hydrophobic phase may comprise at least one hydrophobic substance that is a volatile material. In other cases, only non-volatile hydrophobic substances may be present in the hydrophobic phase.

Suitable hydrophobic substances may include silicones (silicone oils), hydrocarbons, esters, amides, ethers, and mixtures thereof. Suitable hydrocarbons may include straight-chain, branched, or cyclic alkanes and alkenes. The total number of carbon atoms in the hydrocarbon may be selected based on desired functional characteristics such as volatility.

Suitable esters may include those having hydrocarbyl chains derived from fatty acids or alcohols (e.g., monoesters, polyhydric alcohol esters, and di- and tri-carboxylic acid esters). Illustrative esters that may be suitable include, but are not limited to, isopropyl isostearate, hexyl laurate, isohexyl laurate, isohexyl palmitate, isopropyl palmitate, decyl oleate, isodecyl oleate, hexadecyl stearate, decyl stearate, isopropyl isostearate, dihexyldecyl adipate, lauryl lactate, myristyl lactate, cetyl lactate, oleyl stearate, olcyl oleate, oleyl myristate, lauryl acetate, cetyl propionate, C₁₂-C₁₅ alkyl benzoate, butyloctyl salicylate, phenylethyl benzoate, dicaprylyl carbonate, dioctyl malate, dicaprylyl malcate, isononyl isononanoate, propylene glycol dicaprate, diisopropyl adipate, dibutyl adipate, and oleyl adipate. Other esters suitable for use in the cosmetic compositions described herein include those known as polyhydric alcohol esters and glycerides. Still other suitable esters include those described in the Personal Care Product Council's International Cosmetic Ingredient Dictionary and Handbook, Thirteenth Edition, 2010, under the functional category of “Esters.”

Suitable ethers may include saturated and unsaturated fatty ethers of a polyhydric alcohol, and alkoxylated derivatives thereof. Illustrative ethers may include, but are not limited to, C₄₋₂₀ alkyl ethers of polypropylene glycols, and di-C₈₋₃₀ alkyl ethers. Suitable examples of these materials include PPG-14 butyl ether, PPG-15 stearyl ether, PPG-11 stearyl ether, dioctyl ether, dodecyl octyl ether, and mixtures thereof.

Suitable silicones are polysiloxanes. Polysiloxanes may have a viscosity of about 0.5 to about 1,000,000 centistokes (cSt) at 25° C. Such polysiloxanes can be represented by the general chemical formula R₃SiO[R₂SiO]ₓSiR₃, wherein each R is independently hydrogen or a hydrocarbyl group, preferably a C₁-C₃₀, C₁-C₁₀, C₁-C₆, or C₁-C₄ hydrocarbyl group, which may be saturated or unsaturated. The hydrocarbyl group may be a straight-chain, branched, or cyclic alkyl group, an aryl group, or any combination thereof. Variable x represents the degree of polymerization and is an integer ranging from 0 to about 10,000. In certain embodiments, R is hydrogen, methyl, or ethyl, preferably methyl or ethyl. Commercially available polysiloxanes include polydimethylsiloxanes, also known as dimethicones, examples of which include the DM-FLUID series available from Shin-Etsu, the VISCASIL® series available from Momentive Performance Materials Inc., and the DOW CORNING® 200 series (also sold as XIAMETER® PMX-200 Silicone Fluids) available by Dow Corning Corporation. XIAMETER® PMX-200 Silicone Fluids, for example, are available in various grades having room-temperature kinematic viscosity values of 0.65, 1.5, 50, 100, 350, 10,000, 12,500 100,000, and 300,000 cSt.

Substituted dimethicones suitable for use in the hydrophobic phase of the cosmetic compositions disclosed herein may include, for example, phenyl dimethicone (sold as BOTANSIL™ PD-151, available from Botanigenics, Inc.), diphenyl dimethicone (KF-53 and KF-54 available from Shin-Etsu), phenyl trimethicone (DOWSIL™ 556 Cosmetic Grade Fluid, available from Dow Corning), and trimethylsiloxyphenyl dimethicone (BELSIL® PDM-20, BELSIL®PDM-200, or BELSIL®PDM-1000 from Wacker). Other suitable substituted dimethicones may include alkyl dimethicones having at least one fatty alkyl (e.g., a C₁₂-C₂₂ alkyl), such as cetyl dimethicone.

Cyclic silicones may also be suitable for use in the cosmetic compositions of the present disclosure. Suitable cyclic silicones may contain 3-8 repeat units or a mixture thereof. Examples of commercially available cyclic silicones include DOW CORNING® UP-1001 ULTRA PURE FLUID (4 dimethylsiloxane repeat units), XIAMETER® PMX-0245 (5 dimethylsiloxane repeat units), XIAMETER® PMX-0245 (6 dimethylsiloxane repeat units), DOW CORNING® 245 fluid (mixture of 4 and 5 dimethylsiloxane repeat units), and DOW CORNING® 345 fluid (mixture of 4, 5, and 6 dimethylsiloxane repeat units).

The hydrophobic phase may comprise one or more silicones, such as dimethicone or one or more other silicones, in an amount of about 20 wt % or above, or about 30 wt % or above, or about 40 wt % or above, or about 50 wt % or above, based on total mass of the hydrophobic phase. In non-limiting examples, the hydrophobic phase may comprise one or more silicones, such as dimethicone, in an amount ranging from about cl to about c2, based on total mass of the hydrophobic phase, where c1 and c2 may be, independently, 20, 21, 22, 23, 24, 25, 26, 27, 28, 29, or 30, and where c1<c2. For example, the cosmetic compositions may comprise the one or more silicones, such as dimethicone, in an amount ranging from about 20 wt % to about 30 wt %, or about 20 wt % to about 25 wt %, or about 21 wt % to about 26 wt %, or about 22 wt % to about 27 wt %, or about 23 wt % to about 28 wt %, or about 24 wt % to about 29 wt %, or about 25 wt % to about 30 wt %.

Aqueous phases suitable for use in the cosmetic compositions of the present disclosure may comprise an aqueous fluid. Suitable aqueous fluids may comprise water and optionally one or more water-miscible solvents. Suitable water-miscible solvents may include monohydric alcohols, dihydric alcohols, polyhydric alcohols, glycerol, glycols, polyalkylene glycols such as polyethylene glycol, and mixtures thereof. Particularly suitable water-miscible solvents that may be present include lower aliphatic alcohols such as ethanol, propanol, isopropanol, and butanol; diols such as 1,2-propanediol, 1,3-propanediol, 1,3-butanediol, 1,4-butanediol, pentanediol, hexanediol, heptanediol, or decanediol; glycerin; and mixtures thereof. In certain embodiments, the cosmetic compositions disclosed herein may comprise water, at least one diol, glycerin, and any combination thereof in the aqueous phase.

The aqueous phase may comprise water in an amount of about 10 wt % or above, or about 20 wt % or above, or about 30 wt % or above, or about 40 wt % or above, based on total mass of the aqueous phase. In non-limiting examples, the aqueous phase may comprise water in an amount ranging from about d1 to about d2, based on total mass of the aqueous phase, where d1 and d2 may be, independently, 15, 16, 17, 18, 19, 20, 21, 22, 23, 24, 25, 26, 27, 28, 29, or 30, and where d1<d2. For example, the cosmetic compositions may comprise water in an amount ranging from about 16 wt % to about 30 wt %, or about 16 wt % to about 20 wt %, or about 20 wt % to about 25 wt %, or about 25 wt % to about 30 wt %, or about 16 wt % to about 23 wt %, or about 17 wt % to about 24 wt %, or about 18 wt % to about 25 wt %, or about 19 wt % to about 26 wt %, or about 20 wt % to about 27 wt %, or about 21 wt % to about 28 wt %, or about 22 wt % to about 29 wt %, or about 23 wt % to about 30 wt %.

As indicated above, the carboxylic acid-based agent may be present in the aqueous phase of the cosmetic compositions disclosed herein, preferably with the carboxylic acid-based agent being dissolved in the aqueous phase. The carboxylic acid-based agent may be present in the cosmetic compositions in an amount ranging from about e1 to about e2 wt %, based on total mass of the cosmetic composition, where e1 and e2 may independently be 1, 2, 3, 4, 5, 6, 7, 8, 9, or 10, and where e1<c2. In non-limiting examples, the amount of the carboxylic acid-based agent may range from about 1 wt % to about 10 wt %, or about 2 wt % to about 8 wt %, or about 2 wt % to about 6 wt %, or about 3 wt % to about 7 wt %, or about 3 wt % to about 5 wt %, based on total mass of the cosmetic composition.

Suitable carboxylic acid-based agents may comprise a carboxylic acid, any ester thereof, any salt thereof, or any combination thereof. Suitable carboxylic acids may include monocarboxylic acids (e.g., stearic acid, linoleic acid, palmitic acid, oleic acid, and the like), dicarboxylic acids, polycarboxylic acids, amino acids, hydroxyacids, polyhydroxyacids, or any combination thereof. Suitable polycarboxylic acids may include dicarboxylic acids, such as C₂-C₁₀ dicarboxylic acids (e.g., azelaic acid and the like), or carboxylic acid compounds having more than two carboxylic acid groups. Suitable amino acids may include, but are not limited to, tranexamic acid or any salt thereof.

At least a portion of or all of the carboxylic acid-based agent may comprise a hydroxyacid. Suitable hydroxyacids may include alpha-hydroxyacids (i.e., an alcoholic hydroxyl group is located one carbon atom away from the carbonyl of the carboxylic acid group), beta-hydroxyacids (i.e., the alcoholic hydroxyl group is located two carbon atoms away from the carbonyl group of the carboxylic acid group), and polyhydroxyacids having two or more alcoholic hydroxyl groups, which may be located in any open valence position of the carbon chain. Suitable polyhydroxyacids may be alpha-hydroxyacids or beta-hydroxyacids having one or more additional alcoholic hydroxyl groups.

Examples of suitable alpha-hydroxyacids may include, for example, citric acid, tartaric acid, lactic acid, the like, and any combination thereof. Examples of suitable beta-hydroxyacids may include, for example, salicylic acid, carnitine, 2-hydroxybutanoic acid, 3-hydroxypentanoic acid, the like, and any combination thereof. Examples of suitable polyhydroxyacids may include, for example, gluconic acid, mandelic acid, lactobionic acid, the like, and any combination thereof. Any of the foregoing carboxylic acids may be in esterified form when initially formulated in the dispersion medium, and the corresponding carboxylic acid may form in situ through hydrolysis in the dispersion medium. For example, the carboxylic acid-based agent may comprise an ester of a polyhydroxyacid, such as glucono-delta-lactone (i.e., the cyclic ester of gluconic acid), the like, or any combination thereof, and gluconic acid may be released in situ through hydrolysis. Accordingly, carboxylic acid-based agents suitable for use in the cosmetic compositions of the present disclosure may comprise gluconic acid, any ester thereof, any salt thereof, or any combination thereof.

The aqueous phase of the dispersion medium may have a pH value suitable for maintaining the pigment particles stably dispersed in the hydrophobic phase, as well as for providing the carboxylic acid-based agent in a specified form. For example, the pH value of the aqueous phase may promote in situ ester hydrolysis to form the carboxylate salt form of the carboxylic acid-based agent. The aqueous phase of the dispersion medium may have a pH value ranging from about f1 to about f2 wt %, where f1 and f2 can be, independently, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, or 9.0, and where f1<f2. In non-limiting examples, the pH value of the aqueous phase of the dispersion medium may range from about 4 to about 9, or from about 4.5 to about 6, or about 5 to about 6, or about 5 to about 5.5.

A plurality of coated pigment particles may be dispersed as solids in the hydrophobic phase of the dispersion medium. Cosmetic compositions of the present disclosure may comprise a weight percent of coated pigment particles ranging from about g1 to about g2 wt %, based on total mass of the cosmetic composition, where g1 and g2 can be, independently, 1, 2, 3, 4, 5, 6, 7, 8, 9, 10, 11, 12, 13, 14, 15, 16, 17, 18, 19, or 20 and where g1<g2. In non-limiting examples, the coated pigment particles may be present in the cosmetic compositions in an amount ranging from about 1 wt % to about 20 wt %, or about 2 wt % to about 20 wt %, or about 3 wt % to about 18 wt %, or about 4 wt % to about 16 wt %, or about 5 wt % to about 15 wt %, or about 5 wt % to about 10 wt %, based on total mass of the cosmetic compositions.

The coated pigment particles may comprise one type or more than one type of coated pigment particle. Various types of coated pigment particles may have different colors, different particle sizes, different minerals, or any combination thereof, as non-limiting examples. When different types of coated pigment particles are present, the various individual types of coated pigment particles may be present in any ratio with respect to one another. The ratio of the individual types of coated pigment particles may be selected, for example, to convey a specific tint, shade, or coloration to the cosmetic compositions. Individual types of coated pigment particles need not necessarily reside within the coated pigment particle wt % ranges described above, but, in total, the different types of coated pigment particles may collectively reside within the foregoing wt % ranges.

The coated pigment particles used herein may comprise a pigment material that is then coated with a coating material. Suitable pigment particles may include metal oxide pigment particles, metal hydroxide pigment particles, mica pigment particles, talc pigment particles, the like, and any combination thereof.

Metal oxide pigment particles may include, for example, iron (III) oxide, titanium (IV) oxide, zinc oxide, chromium (III) oxide, or any combination thereof. For purposes of this disclosure, silicate minerals, such as mica and talc, are not considered to be metal oxides. Metal hydroxide pigment particles may include, for example, aluminum hydroxide, the like, or any combination thereof. Non-limiting examples of metal oxide pigment particles may include, for instance, iron oxide pigment particles such as iron oxide red, iron oxide yellow, and iron oxide black, or any combination thereof. Such iron oxide pigment particles may comprise iron (III) oxide, which is coated with a coating material. In some cases, suitable pigment particles may include iron (III) oxide pigment particles admixed with titanium dioxide pigment particles. In some cases, suitable pigment particles may include iron (III) oxide pigment particles admixed with mica pigment particles.

If metal oxide pigment particles are present in combination with other types of pigment particles, the metal oxide pigment particles may comprise a majority (>50% on a mass basis) of the total pigment particles that are present in the cosmetic compositions. For example, iron oxide pigment particles (e.g., iron (III) oxide pigment particles having a coating) may be present in combination with other types of pigment particles, wherein the iron oxide pigment particles comprise about 50 wt % or above, or about 60 wt % or above, or about 70 wt % or above, or about 80 wt % or above, or about 90 wt % or above of the total pigment particles present in the cosmetic compositions, and the other types of pigment particles are present in a non-zero amount of the total pigment particles, such that the iron oxide pigment particles and the other types of pigment particles total 100% of the total pigment particles present in the cosmetic compositions.

Coated pigment particles suitable for use in the cosmetic compositions of the present disclosure may comprise various types of coatings. Suitable coatings for use in coated pigment particles may include, for example, silicon-based coatings, alkylester-based coatings, alkylaminoacid-based coatings, and any combination thereof.

In non-limiting embodiments, the coating upon the coated pigment particles may comprise a silicon-based coating, which may comprise a silica coating. The silica coating may be present in an amount ranging from about h1 to about h2 wt %, based on total mass of the coated pigment particles, where h1 and h2 can be, independently, 5, 6, 7, 8, 9, 10, 11, 12, 13, 14, 15, 16, 17, 18, 19, or 20, and where h1<h2. In non-limiting examples, the coated pigment particles may contain the silica coating in an amount ranging from about 5 wt % to about 20 wt %, or about 8 wt % to about 16 wt %, or about 10 wt % to about 20 wt %, or about 10 wt % to about 15 wt %, based on total mass of the coated pigment particles.

Examples of suitable commercial coated pigment particles having a silica coating may include PRIMROSE silica-coated iron oxide pigments available from Miyoshi America, Inc.

Optionally, coated pigment particles having a silica coating may be combined with other types of coated pigment particles not having a silica coating. When combined with other types of coated pigment particles not having a silica coating, the coated pigment particles may comprise a majority of the coated pigment particles on a mass basis.

Other types of coated pigment particles may have an organic-based coating. Such coated pigment particles having an organic-based coating may be present as a minority component of the total coated pigment particles in combination with coated pigment particles having a silica coating. In non-limiting examples, coated pigment particles having an organic-based coating may be present in an amount ranging from about 0.1 wt % to about 25 wt %, or about 0.5 to about 20 wt %, or about 1 wt % to about 18 wt %, or about 10 wt % to about 16 wt %, based upon total mass of the coated pigment particles.

Suitable organic-based coatings may include an organosilicon-based material, an organoester-based material, an organoaminoacid-based material, the like, or any combination thereof. Organosilicon-based materials may include, for example, a dimethicone material, an alkylsiloxysilicate material (e.g., a trimethylsiloxysilicate material), an alkylsilane material (e.g., a tricthoxycaprylylsilane material), the like, and any combination thereof.

In addition to providing coloration and promoting exfoliation of the skin, another common use for cosmetic compositions may include address development of hyperpigmented spots on the skin. Hyperpigmentation may result due to aging, sun exposure, or post-inflammation, the latter being referred to as PIH or post-inflammatory hyperpigmentation.

The cosmetic compositions of the present disclosure may further comprise a skin-lightening agent, such as niacinamide (also referred to as nicotinamide). Advantageously, the pH range of the aqueous phase of the dispersion medium may be suitable for promoting stability of niacinamide. Niacinamide exhibits suitable stability within a pH range of about 5 to about 7. Outside of this pH range, niacinamide may undergo hydrolysis to nicotinic acid, a chemical which may result in skin flushing and irritation.

When present, niacinamide or other suitable skin-lightening agents may be present in the cosmetic compositions disclosed herein in an amount suitable to promote skin lightening. Preferably, the niacinamide is located in the aqueous phase of the dispersion medium. In non-limiting examples, the cosmetic compositions of the present disclosure may comprise niacinamide in an amount ranging from about i1 to about i2 wt %, based on total mass of the cosmetic composition, where i1 and i2 can be, independently, 1, 2, 3, 4, 5, 6, 7, 8, 9, or 10, and where i1<i2. In non-limiting examples, the cosmetic compositions of the present disclosure may comprise niacinamide or another suitable skin-lightening agent in an amount ranging from about 1 wt % to about 10 wt %, or about 2 wt % to about 8 wt %, or about 3 wt % to about 5 wt %, based on total mass of the cosmetic composition.

Other suitable skin-lightening agents, which may be present alone or in addition to niacinamide, include, for example, Vitamin C, kojic acid, tranexamic acid, ferulic acid, licorice extract, resorcinols (e.g., phenylethyl resorcinol, hexylresorcinol, and the like), alpha-arbutin, glutathione, hydroquinone, any derivative thereof, or any combination thereof.

Cosmetic compositions of the present disclosure, including those comprising niacinamide, may be substantially free of nicotinic acid. As used herein, the term “substantially free,” and grammatical variants thereof, refer to quantities of nicotinic acid that are about 0.1 wt % or below, preferably about 0.01 wt % or below, and more preferably 0.001 wt % or below, based upon total mass of the cosmetic compositions. The cosmetic compositions may be maintained substantially free of nicotinic acid by keeping the aqueous phase of the dispersion medium within a pH range at which nicotinic acid does not readily form from niacinamide.

In more particular embodiments, cosmetic compositions of the present disclosure may comprise: a dispersion medium comprising an aqueous phase and a hydrophobic phase; wherein the aqueous phase has a pH ranging from about 5 to about 5.5; wherein the dispersion medium comprises a water-in-oil emulsion comprising the aqueous phase as a dispersed phase and the hydrophobic phase as a continuous phase; about 1 wt % to about 10 wt % of gluconic acid, any ester thereof, any salt thereof, or any combination thereof dispersed in the aqueous phase of the dispersion medium, based upon total mass of the cosmetic composition; about 5 wt % to about 15 wt % of a plurality of coated pigment particles dispersed as solids in the hydrophobic phase of the dispersion medium, based upon total mass of the cosmetic composition; wherein the coated pigment particles comprise a silica coating; and about 1 wt % to about 10 wt % of niacinamide dispersed in the dispersion medium, based upon total mass of the cosmetic composition.

Cosmetic compositions of the present disclosure may have various chemical or physical properties. Cosmetic compositions of the present disclosure may have a viscosity ranging from about 10,000 to about 80,000 centipoise (cP), as measured with a Brookfield viscometer using a LVTF spindle at 12 rpm at 25° C. A rheometer (e.g., DHR-2 rheometer from TA Instrument) may be used to measure small amplitude oscillation shear with constant strain of 0.6% at room temperature to determine viscoelastic behavior of the cosmetic compositions of the present disclosure. Cosmetic compositions of the present disclosure may have one or more of: a storage modulus (G′) from about 10 Pascal (Pa) to about 10,000 Pa, and/or a linear viscoelastic region (stress) from about 10 Pa to about 600 Pa. A rheometer (e.g., DHR-2 rheometer from TA Instrument) may be used to measure flow sweep viscosity at room temperature at shear rates of about 0.011 s⁻¹ to 600 s⁻¹. Cosmetic compositions of the present disclosure may have flow sweep viscosities of about 170,000 cP to about 1,700,000 cP at rest (shear rate of 0.01 s⁻¹) and about 350 cP to about 450 cP at high shear (shear rate of 600 s⁻¹).

Cosmetic compositions of the present disclosure may be stable, i.e., substantially unaltered in chemical state, physical homogeneity, and/or color, upon exposure to conditions reasonably expected to be incurred in shipping, storage and use, for example, for at least 30 days at a temperature of from about 0° C. to about 40° C. In certain embodiments, cosmetic compositions of the present disclosure may be stable upon exposure to 30 days at a temperature of about 50° C. and a relative humidity (RH) of about 70 RH. Cosmetic compositions of the present disclosure may be resistant to phase separation, discoloration, settling, odor, gassing, and any combination thereof, upon exposure to 30 days at a temperature of about 50° C. and a relative humidity (RH) of about 70 RH. Physical parameters associated with the testing conditions may change by about 15% or less over the testing period, preferably by about 5% or less over the testing period, and more preferably by about 1% or less over the testing period.

Cosmetic compositions of the present disclosure may be color stable, i.e., stable against color shift upon exposure to 30 days at room temperature or elevated temperature (e.g., about 40° C. to about 50° C.), and at a relative humidity (RH) of about 70 RH. Color shift may be measured using a spectrophotometer (e.g., an X-RITE MAT-12 spectrophotometer). Color shift may be reported according to the L*a*b* color scale, where L* is darkness to lightness scale (higher is lighter, lower is darker), a* is green to red scale (more positive is more red, more negative is more green, and zero is neutral), and b* is blue to yellow scale (more positive is more yellow, more negative is more blue, and zero is neutral), and ΔE_{cme} is a composite of the difference between L*, a*, and b* values from the original measurement. Acceptability criteria for ΔE_{cme} may be ≤2. In certain embodiments, cosmetic compositions of the present disclosure, following exposure to stability testing conditions, may have a pH of the aqueous phase of the dispersion medium of about 4.5 to about 6.0, a viscosity of about 23,000 cP (Brookfield viscometer, LVTF spindle, 12 rpm, 1 minute), and a ΔE_{cme} of less than 1 after storage at 40° C. for about 3 months, compared to an initial time period, room temperature control.

It is to be appreciated that the cosmetic compositions of the present disclosure may comprise one or more additional ingredients suitable for use in cosmetic compositions and that do not interfere with the ability of the carboxylic acid-based agents to promote exfoliation, the ability of niacinamide to promote skin lightening, and/or the ability of the hydrophobic phase to disperse the coated pigment particles as solids under typical production storage conditions. Non-limiting examples of such additional ingredients may include, but are not limited to, preservatives, antioxidants, pH adjusters, solvents, oils, humectants, moisturizers, conditioning agents, surfactants, emulsifiers, thickeners, vitamins, antioxidants, peptides, fragrances, minerals, colorants, waxes, clays, elastomers (including elastomer gels), particulates (such as mica and/or silica), sunscreens (physical or chemical), and inert ingredients. Examples of such additional ingredients will be familiar to persons having ordinary skill in the art of formulating cosmetic compositions.

The cosmetic compositions of the present disclosure may be applied topically to the skin as part of a user's skincare routine. Optionally, the user may have previously determined that they have a deficiency of skin exfoliation and/or hyperpigmented skin, or suspect that they have such condition(s). The cosmetic compositions may be applied to the skin in a desired location one or more times a day, such as in the morning or in the evening, such as after moisturizing the skin.

Methods for preparing the cosmetic compositions may take place by blending a first dispersion containing the coated pigment particles with a second dispersion comprising the carboxylic acid-based agent. Preferably, the coated pigment particles are dispersed as solids in the first dispersion and the carboxylic acid-based agent is at least partially dissolved in the second dispersion, preferably fully dissolved as a solution in the second dispersion.

More specifically, methods for forming the cosmetic compositions of the present disclosure may comprise: providing a first dispersion comprising a plurality of coated pigment particles dispersed as solids in a hydrophobic phase; wherein the coated pigment particles comprise a silica coating; blending the first dispersion with a second dispersion comprising a carboxylic acid-based agent comprising at least one carboxylic acid, any ester thereof, any salt thereof, or any combination thereof dissolved in an aqueous phase; wherein the second dispersion has a pH ranging from about 4 to about 9; and obtaining a dispersion medium comprising the aqueous phase as a dispersed phase and the hydrophobic phase as a continuous phase; wherein, in the dispersion medium, the carboxylic acid-based agent comprising at least one carboxylic acid, any ester thereof, any salt thereof, or any combination thereof remains dissolved in the aqueous phase, and the coated pigment particles remain dispersed as solids in the hydrophobic phase.

In forming the dispersion medium, at least one of the first dispersion or the second dispersion may comprise niacinamide. In some examples, the niacinamide may be pre-dissolved in water, and then added to the hydrophobic phase.

After the dispersion medium containing the coated pigment particles and the carboxylic acid-based agent has been prepared, the pH value of the aqueous phase of the dispersion medium may be adjusted to about 5 to about 5.5.

Embodiments disclosed herein include the following:
Embodiment 1. A cosmetic composition comprising: a dispersion medium comprising an aqueous phase and a hydrophobic phase; a carboxylic acid-based agent comprising at least one carboxylic acid, any ester thereof, any salt thereof, or any combination thereof dispersed in the aqueous phase of the dispersion medium; and a plurality of coated pigment particles dispersed as solids in the hydrophobic phase of the dispersion medium.
Embodiment 2. The cosmetic composition of Embodiment 1, wherein the aqueous phase is a dispersed phase of the dispersion medium and the hydrophobic phase is a continuous phase of the dispersion medium.
Embodiment 3. The cosmetic composition of Embodiment 1 or Embodiment 2, wherein the dispersion medium comprises a water-in-oil emulsion.
Embodiment 4. The cosmetic composition of any one of Embodiments 1-3, wherein the carboxylic acid-based agent comprises gluconic acid, mandelic acid, azelaic acid, tranexamic acid, lactobionic acid, or any combination thereof.
Embodiment 5. The cosmetic composition of any one of Embodiments 1-4, wherein the carboxylic acid-based agent comprises gluconic acid, any ester thereof, any salt thereof, or any combination thereof.
Embodiment 6. The cosmetic composition of any one of Embodiments 1-5, wherein the coated pigment particles comprise a silica coating.
Embodiment 7. The cosmetic composition of any one of Embodiments 1-6, wherein coated pigment particles comprise iron (III) oxide pigment particles.
Embodiment 8. The cosmetic composition of Embodiment 6 or Embodiment 7, wherein the coated pigment particles comprise about 10 wt % to about 20 wt % of the silica coating, based upon total mass of the coated pigment particles.
Embodiment 9. The cosmetic composition of any one of Embodiments 1-8, further comprising: niacinamide dispersed in the dispersion medium.
Embodiment 10. The composition of claim 9, wherein the cosmetic composition comprises about 1 wt % to about 10 wt % of the niacinamide, based upon total mass of the cosmetic composition.
Embodiment 11. The cosmetic composition of any one of Embodiments 1-10, wherein the aqueous phase of the dispersion medium has a pH ranging from about 5 to about 5.5.
Embodiment 12. The cosmetic composition of any one of Embodiments 1-11, wherein the cosmetic composition comprises about 2 wt % to about 20 wt % of the coated pigment particles, based upon total mass of the cosmetic composition.
Embodiment 13. The cosmetic composition of any one of Embodiments 1-12, wherein the cosmetic composition comprises about 1 wt % to about 10 wt % of the carboxylic acid-based agent, based upon total mass of the cosmetic composition.
Embodiment 14. A cosmetic composition comprising: a dispersion medium comprising an aqueous phase and a hydrophobic phase, the aqueous phase having a pH ranging from about 5 to about 5.5; wherein the dispersion medium comprises a water-in-oil emulsion comprising the aqueous phase as a dispersed phase and the hydrophobic phase as a continuous phase; about 1 wt % to about 10 wt % of gluconic acid, any ester thereof, any salt thereof, or any combination thereof dispersed in the aqueous phase of the dispersion medium, based upon total mass of the cosmetic composition; about 2 wt % to about 20 wt % of a plurality of coated pigment particles dispersed as solids in the hydrophobic phase of the dispersion medium, based upon total mass of the cosmetic composition; wherein the coated pigment particles comprise a silica coating; and about 1 wt % to about 10 wt % of niacinamide dispersed in the dispersion medium, based upon total mass of the cosmetic composition.
Embodiment 15. The cosmetic composition of Embodiment 14, wherein the cosmetic composition is substantially free of nicotinic acid.
Embodiment 16. The cosmetic composition of Embodiment 14 or Embodiment 15, wherein coated pigment particles comprise iron (III) oxide pigment particles.
Embodiment 17. The cosmetic composition of any one of Embodiments 14-16, wherein the coated pigment particles comprise about 10 wt % to about 20 wt % of the silica coating, based upon total mass of the coated pigment particles.
Embodiment 18. A method comprising: providing a first dispersion comprising a plurality of coated pigment particles dispersed as solids in a hydrophobic phase; wherein the coated pigment particles comprise a silica coating; blending the first dispersion with a second dispersion comprising a carboxylic acid-based agent, any ester thereof, any salt thereof, or any combination thereof dispersed in an aqueous phase; wherein the second dispersion has a pH ranging from about 4 to about 9.0; and obtaining a dispersion medium comprising the aqueous phase as a dispersed phase and the hydrophobic phase as a continuous phase; wherein, in the dispersion medium, the carboxylic acid-based agent, any ester thereof, any salt thereof, or any combination thereof remains in the aqueous phase, and the coated pigment particles remain dispersed as solids in the hydrophobic phase.
Embodiment 19. The method of Embodiment 18, wherein at least one of the first dispersion or the second dispersion comprises niacinamide.
Embodiment 20. The method of Embodiment 18 or Embodiment 19, further comprising: adjusting a pH value of the aqueous phase to about 5 to about 5.5.

To facilitate a better understanding of the present disclosure, the following examples of preferred or representative embodiments are given. In no way should the following examples be read to limit, or to define, the scope of the invention.

### Examples

Experimental Details. The following examples were performed using water-in-oil emulsions each comprising approximately a 2:1 mass ratio of a hydrophobic phase to aqueous phase. The foregoing mass ratio is inclusive of all components dissolved or dispersed in each respective phase, including at least dimethicone and coated pigment particles in the hydrophobic phase, and water, glucono-delta-lactone, and niacinamide in the aqueous phase.

The coated pigment particles were silica-coated PRIMROSE iron (III) oxide pigments (Miyoshi America, Inc.).

Alternative coated pigments that were explored included Gelest trimethylsiloxysilicate-coated pigments, Terra mica pigments coated with jojoba ester, and NAID vegetable amino acid treated pigments.

Coated iron (III) oxide pigments were present at about 4.5-10 wt % with respect to the total mass of the water-in-oil emulsion, and at about 6.7-15 wt % with respect to mass of the hydrophobic phase. The hydrophobic phase contained dimethicone in an amount of about 16 wt %, based on total mass of the water-in-oil emulsion. Other oil-based additives in the hydrophobic phase included sunscreens, texturizing agents, dispersants, and other oil-based actives.

The aqueous phase contained water in an amount of about 16 wt %, based on the total mass of the water-in-oil emulsion. Glucono-delta-lactone (as gluconic acid) was present at about 4.5 wt % with respect to total mass of the water-in-oil emulsion, and at about 13.5 wt % with respect to the aqueous phase. Niacinamide was present at about 4 wt % with respect to total mass of the water-in-oil emulsion, and at about 12% with respect to the aqueous phase. Other hydrophilic additives in the aqueous phase included water, water-miscible solvents (e.g., glycerol, glycols, and the like), and water-based additives (e.g., base, surfactants, water-based actives). The pH of the aqueous phase was adjusted to about 5.2-5.3 for analysis.

Shaker Testing. Shaker testing was performed to simulate shipping. Shaker testing was performed using EXCELLA E24 Incubator Shaker Series by New Brunswick Scientific. In brief, various samples (see below) were kept in the shaker for 8 hours at 40° C. shaking at 250 rotations per minute (rpm). Samples were evaluated for phase separation, sedimentation, and color striation. Samples were evaluated and graded. A score of FAIL was given to samples showing separation, color striation, or sedimentation. A score of ACCEPTABLE was given to samples with a homogenous appearance with extremely minor separation or color striation. A score of PASS was given to samples with an unchanged appearance from the original state.

Table 1 shows the shaker test results for various water-in-oil emulsion samples. The most stable samples contained glucono-delta-lactone (GDL) and PRIMROSE pigments. The most stable pH value of the aqueous phase in the absence of niacinamide (NIA) was about 4 to about 4.5, while the most stable pH value of the aqueous phase in the presence of niacinamide was about 5.2. The wt % values in Table 1 are with respect to the total mass of the oil-in-water emulsion.

TABLE 1

| | | | | | | | Shaker |
|---|---|---|---|---|---|---|---|
| | Agent | | Pigment | | NIA | | Test |
| Entry | Type | Wt % | Type | Wt % | Wt % | pH | Results |
| 1 | GDL | 4.5 | PRIMROSE | 10 | 0 | 4.25 | PASS |
| 2 | GDL | 4.5 | PRIMROSE | 10 | 0 | 4.40 | PASS |
| 3 | GDL | 4.5 | PRIMROSE/ | 7/6 | 0 | 4.50 | PASS |
| | | | TERRA NJE5 | | | | |
| 4 | GDL | 4.5 | GELEST/ | 7/6 | 0 | N/A | ACCEPTABLE |
| | | | TERRA NJE5 | | | | |
| 5 | GDL | 4.5 | PRIMROSE | 10 | 2.0 | 4.02 | ACCEPTABLE |
| 6 | GDL | 4.5 | PRIMROSE | 10 | 4.5 | 5.2 | PASS |

Stability Testing. Stability testing was performed to simulate shelf life. Stability testing was performed in a temperature/humidity-controlled chamber. In brief, various samples (see below) were placed in room temperature, 4° C., 40° C., 45° C., 50° C., Cycle, Freeze/Thaw, light box, and Q-SUN for up to 1 year. The light box test is to mimic store light, and Q-SUN is to mimic natural sun exposure. The samples were evaluated for phase separation, sedimentation, color striation, and viscosity changes. Samples were evaluated and graded. A score of FAIL was given to samples in which phase separation (with changes in viscosity), gassing, or color striation was observed throughout the test. A score of ACCEPTABLE was given to samples in which there were no indications of phase separation, viscosity, gassing, or color striation. A score of PASS was given to samples in which no separation, gassing, color striation, and very small change in viscosity were observed throughout the test. Table 2 shows stability test results for various water-in-oil emulsion samples (* indicates stability testing evaluated at 24+ weeks). The wt % values in Table 2 are with respect to the total mass of the oil-in-water emulsion.

TABLE 2

| | | | | | | | Stability |
|---|---|---|---|---|---|---|---|
| | Agent | | Pigment | | NIA | | Test |
| Entry | Type | Wt % | Type | Wt % | Wt % | pH | Results |
| 1 | GDL | 4.5 | PRIMROSE | 10 | 0 | 4.25 | PASS |
| 2 | GDL | 4.5 | PRIMROSE | 10 | 0 | 4.40 | PASS |
| 3 | GDL | 4.5 | PRIMROSE/ | 7/6 | 0 | 4.50 | FAIL |
| | | | TERRA NJE5 | | | | |
| 4 | GDL | 4.5 | GELEST/ | 7/6 | 0 | N/A | FAIL |
| | | | TERRA NJE5 | | | | |
| 5 | GDL | 4.5 | PRIMROSE | 10 | 2.0 | 4.02 | PASS* |
| | | | | | | | 24+ weeks |
| 6 | GDL | 4.5 | PRIMROSE | 10 | 4.5 | 5.2 | PASS* |
| | | | | | | | 24+ weeks |

Since the water-in-oil emulsions containing only PRIMROSE pigments passed both the shaker test and the stability test, these were the coated pigments explored in subsequent experiments.

Sweep Testing. Sweep testing was performed to simulate shade matching. Sweep testing was performed using a Caframo mixer with a U-shaped mixing blade. The samples were evaluated for phase separation, sedimentation, viscosity changes, and color striation. Samples were evaluated and graded. A score of FAIL was given to samples in which separation, sedimentation, color striation, or a decrease in viscosity occurred. A score of ACCEPTABLE was given to samples in which no separation, sedimentation, color striation, and very slight change in viscosity was observed. A score of PASS was given to samples in which no separation, sedimentation, color striation, and change in viscosity was observed. Table 3 shows the sweep test performance for various water-in-oil emulsion samples. The wt % values in Table 3 are with respect to the total mass of the oil-in-water emulsion.

TABLE 3

| | Agent | | Pigment | | NIA | | Sweep Test |
|---|---|---|---|---|---|---|---|
| Entry | Type | Wt % | Type | Wt % | Wt % | pH | Results |
| 1 | GDL | 4.5 | PRIMROSE | 10 | 0 | 4.20 | PASS |
| 2 | GDL | 4.5 | PRIMROSE | 10 | 0 | 4.50 | PASS |

Niacinamide Degradation Testing. Niacinamide degradation testing was performed to understand the rate of niacinamide degradation into nicotinic acid. Niacinamide degradation testing was performed using ultra performance liquid chromatography (UPLC) and liquid chromatography-mass spectrometry (LC-MS) test methods. UPLC was used to analyze for niacinamide, and LC-MS was used to analyze nicotinic acid. Water-in-oil emulsion cosmetic composition samples with 4.5 wt. % of GDL, 10 wt % of PRIMROSE coated pigment particles, ˜4 wt % of niacinamide, and a pH of 5.26 were evaluated for nicotinic acid concentration at several time points (initial, 1 month, 2 months, and 3 months). Testing was performed in a temperature/humidity-controlled chamber for 3 months at room temperature testing conditions (25° C.±2° C., 60% relative humidity (RH)±5% RH) and at accelerated temperature testing conditions (40° C.±2° C., 60% RH±5% RH). UPLC and LC-MS analyses were performed on the samples every month. Concentrations of niacinamide and nicotinic acid in the samples with respect to the total mass of the water-in-oil emulsion over time are shown in Table 4.

TABLE 4

| Conditions | |
|---|---|
| Niacinamide Wt % | Nicotinic Acid Wt % |

| Time Points | 25° C. | 40° C. | 25° C. | 40° C. |
|---|---|---|---|---|
| Initial | 4.21 | 4.21 | 0.001 | 0.001 |

| | | | | | |
|---|---|---|---|---|---|
| 1 | Month | 4.20 | 4.19 | 0.01 | 0.03 |
| 2 | Months | 4.26 | 4.21 | 0.01 | 0.06 |
| 3 | Months | 4.17 | 4.12 | 0.01 | 0.08 |

In vitro Melanin Testing. An in vitro study was conducted using a MELANODERM (MatTek) assay to assay melanin production by the cosmetic compositions. A control sample was assigned a relative melanin value of 1.0. The amount of melanin for a fully formulated sample (Table 1, Entry 6) was measured relative to the control. A first placebo sample lacking niacinamide and glucono-delta-lactone and a second placebo sample further lacking all active ingredients were also formulated and measured relative to the control. Testing results are shown in Table 5 below. As shown, after 14 days, the full formula reduced normalized melanin (normalized to viability) by approximately 30.12%, which is indicative of a significant brightening effect.

TABLE 5

| | Melanin in |
|---|---|
| Sample | MELANODERM |
| Control | 1.0000 |
| Placebo 1 | 0.9700 |
| Placebo 2 | 1.1960 |
| Full Formula | 0.6988 |

Color Testing. Color testing was conducted to evaluate the color stability of the cosmetic compositions. Testing was performed in a temperature/humidity-controlled chamber for 3 months at room temperature testing conditions (25° C.±2° C., 60% relative humidity (RH)±5% RH) and at accelerated temperature testing conditions (40° C.±2° C., 60% RH±5% RH). Color shift was measured using a spectrophotometer (e.g., an X-RITE MAT-12 spectrophotometer) and is reported according to the L*a*b* color scale, where L* is darkness to lightness scale (higher is lighter, lower is darker), a* is green to red scale (more positive is more red, more negative is more green, and zero is neutral), and b* is blue to yellow scale (more positive is more yellow, more negative is more blue, and zero is neutral), and ΔE_{cme} is a composite of the difference between L*, a*, and b* values relative to the initial measurement and represents color sensation difference relative to the control. The acceptability criteria for ΔE_{cmc} is ≤2. Testing results are shown in Table 6.

TABLE 6

| Temperature | Time Point | L* | a* | b* | ΔE_{cmc} |
|---|---|---|---|---|---|
| 25° C. | Initial | 27.73 | 13.24 | 8.80 | N/A |
| 25° C. | 3 weeks | 28.06 | 13.20 | 8.73 | 0.23 |
| 40° C. | 3 weeks | 28.19 | 13.34 | 8.90 | 0.32 |
| 25° C. | 9 weeks | 28.00 | 13.22 | 8.82 | 0.18 |
| 40° C. | 9 weeks | 27.90 | 13.28 | 8.95 | 0.19 |
| Room Temperature | 13 weeks | 28.25 | 13.26 | 8.96 | 0.38 |
| 40° C. | 13 weeks | 28.02 | 13.18 | 8.87 | 0.22 |

All documents described herein are incorporated by reference herein for purposes of all jurisdictions where such practice is allowed, including any priority documents and/or testing procedures to the extent they are not inconsistent with this text. As is apparent from the foregoing general description and the specific embodiments, while forms of the disclosure have been illustrated and described, various modifications can be made without departing from the spirit and scope of the disclosure. Accordingly, it is not intended that the disclosure be limited thereby. For example, the compositions described herein may be free of any component, or composition not expressly recited or disclosed herein. Any method may lack any step not recited or disclosed herein. Likewise, the term “comprising” is considered synonymous with the term “including.” Whenever a method, composition, element, or group of elements is preceded with the transitional phrase “comprising,” it is understood that we also contemplate the same composition or group of elements with transitional phrases “consisting essentially of,” “consisting of,” “selected from the group consisting of,” or “is” preceding the recitation of the composition, element, or elements and vice versa.

Unless otherwise indicated, all numbers expressing quantities of ingredients, properties such as molecular weight, reaction conditions, and so forth used in the present specification and associated claims are to be understood as being modified in all instances by the term “about.” Accordingly, unless indicated to the contrary, the numerical parameters set forth in the following specification and attached claims are approximations that may vary depending upon the desired properties sought to be obtained by the embodiments of the present invention. At the very least, and not as an attempt to limit the application of the doctrine of equivalents to the scope of the claim, each numerical parameter should at least be construed in light of the number of reported significant digits and by applying ordinary rounding techniques.

Whenever a numerical range with a lower limit and an upper limit is disclosed, any number and any included range falling within the range is specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values. Also, the terms in the claims have their plain, ordinary meaning unless otherwise explicitly and clearly defined by the patentee. Moreover, the indefinite articles “a” or “an,” as used in the claims, are defined herein to mean one or more than one of the element that it introduces.

One or more illustrative embodiments are presented herein. Not all features of a physical implementation are described or shown in this application for the sake of clarity. It is understood that in the development of a physical embodiment of the present disclosure, numerous implementation-specific decisions must be made to achieve the developer's goals, such as compliance with system-related, business-related, government-related and other constraints, which vary by implementation and from time to time. While a developer's efforts might be time-consuming, such efforts would be, nevertheless, a routine undertaking for one of ordinary skill in the art and having benefit of this disclosure.

Therefore, the present disclosure is well adapted to attain the ends and advantages mentioned as well as those that are inherent therein. The particular embodiments disclosed above are illustrative only, as the present disclosure may be modified and practiced in different but equivalent manners apparent to one having ordinary skill in the art and having the benefit of the teachings herein. Furthermore, no limitations are intended to the details of construction or design herein shown, other than as described in the claims below. It is therefore evident that the particular illustrative embodiments disclosed above may be altered, combined, or modified and all such variations are considered within the scope and spirit of the present disclosure. The embodiments illustratively disclosed herein suitably may be practiced in the absence of any element that is not specifically disclosed herein and/or any optional element disclosed herein.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross-referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests, or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is the following:

1. A cosmetic composition comprising:
a dispersion medium comprising an aqueous phase and a hydrophobic phase;
a carboxylic acid-based agent comprising at least one carboxylic acid, any ester thereof, any salt thereof, or any combination thereof dispersed in the aqueous phase of the dispersion medium; and
a plurality of coated pigment particles dispersed as solids in the hydrophobic phase of the dispersion medium.

2. The cosmetic composition of claim 1, wherein the aqueous phase is a dispersed phase of the dispersion medium and the hydrophobic phase is a continuous phase of the dispersion medium.

3. The cosmetic composition of claim 1, wherein the dispersion medium comprises a water-in-oil emulsion.

4. The cosmetic composition of claim 1, wherein the carboxylic acid-based agent comprises gluconic acid, mandelic acid, azelaic acid, tranexamic acid, lactobionic acid, or any combination thereof.

5. The cosmetic composition of claim 1, wherein the carboxylic acid-based agent comprises gluconic acid, any ester thereof, any salt thereof, or any combination thereof.

6. The cosmetic composition of claim 1, wherein the coated pigment particles comprise a silica coating.

7. The cosmetic composition of claim 6, wherein coated pigment particles comprise iron (III) oxide pigment particles.

8. The cosmetic composition of claim 7, wherein the coated pigment particles comprise about 10 wt % to about 20 wt % of the silica coating, based upon total mass of the coated pigment particles.

9. The cosmetic composition of claim 1, further comprising:
niacinamide dispersed in the aqueous phase of the dispersion medium.

10. The composition of claim 9, wherein the cosmetic composition comprises about 1 wt % to about 10 wt % of the niacinamide, based upon total mass of the cosmetic composition.

11. The cosmetic composition of claim 1, wherein the aqueous phase of the dispersion medium has a pH ranging from about 5 to about 5.5.

12. The cosmetic composition of claim 1, wherein the cosmetic composition comprises about 2 wt % to about 20 wt % of the coated pigment particles, based upon total mass of the cosmetic composition.

13. The cosmetic composition of claim 1, wherein the cosmetic composition comprises about 1 wt % to about 10 wt % of the carboxylic acid-based agent, based upon total mass of the cosmetic composition.

14. A cosmetic composition comprising:
a dispersion medium comprising an aqueous phase and a hydrophobic base, the aqueous phase having a pH ranging from about 5 to about 5.5;
wherein the dispersion medium comprises a water-in-oil emulsion comprising the aqueous phase as a dispersed phase and the hydrophobic phase as a continuous phase;
about 1 wt % to about 10 wt % of gluconic acid, any ester thereof, any salt thereof, or any combination thereof dispersed in the aqueous phase of the dispersion medium, based upon total mass of the cosmetic composition;
about 2 wt % to about 20 wt % of a plurality of coated pigment particles dispersed as solids in the hydrophobic phase of the dispersion medium, based upon total mass of the cosmetic composition;
wherein the coated pigment particles comprise a silica coating; and
about 1 wt % to about 10 wt % of niacinamide dispersed in the aqueous phase of the dispersion medium, based upon total mass of the cosmetic composition.

15. The cosmetic composition of claim 14, wherein the cosmetic composition is substantially free of nicotinic acid.

16. The cosmetic composition of claim 14, wherein coated pigment particles comprise iron (III) oxide pigment particles.

17. The cosmetic composition of claim 16, wherein the coated pigment particles comprise about 10 wt % to about 20 wt % of the silica coating, based upon total mass of the coated pigment particles.

18. A method comprising:
providing a first dispersion comprising a plurality of coated pigment particles dispersed as solids in a hydrophobic phase;
wherein the coated pigment particles comprise a silica coating;
blending the first dispersion with a second dispersion comprising a carboxylic acid-based agent, any ester thereof, any salt thereof, or any combination thereof dispersed in an aqueous phase;
wherein the second dispersion has a pH ranging from about 4 to about 9.0; and
obtaining a dispersion medium comprising the aqueous phase as a dispersed phase and the hydrophobic phase as a continuous phase;
wherein, in the dispersion medium, the carboxylic acid-based agent, any ester thereof, any salt thereof, or any combination thereof remains in the aqueous phase, and the coated pigment particles remain dispersed as solids in the hydrophobic phase.

19. The method of claim 18, wherein at least one of the first dispersion or the second dispersion comprises niacinamide, or wherein niacinamide is added to the dispersion medium.

20. The method of claim 18, further comprising:
adjusting a pH value of the aqueous phase of the dispersion medium to about 5 to about 5.5.
